(19) United States
(12) Patent Application Publication
(10) Pub. No.: US 2021/0219973 A1
(43) Pub. Date: Jul. 22, 2021

(54) SKIN WOUND CLOSING APPARATUS

(71) Applicant: Shanghai Unitedheart Medical Technology Co., Ltd., Shanghai City (CN)

(72) Inventors: JINGXI ZHANG, Shanghai City (CN); YIYANG HUANG, Shanghai City (CN); DAN QU, Shanghai City (CN); YUE HUANG, Shanghai City (CN)

(21) Appl. No.: 17/152,803

(22) Filed: Jan. 20, 2021

(30) Foreign Application Priority Data
Jan. 21, 2020 (CN) 2020100685926
Jan. 21, 2020 (CN) CN2020100685574
Jan. 22, 2020 (CN) 2020201432501
Jan. 22, 2020 (CN) 2020201441939

(51) Int. Cl. A61B 17/04; A61B 17/06

(52) U.S. Cl. CPC A61B 17/0466; A61B 2017/06009; A61B 2017/06014; A61B 17/06004

(57) ABSTRACT

The invention provided with a skin wound closing apparatus which belongs to medical equipment. The skin wound closing apparatus provided in this invention includes: a pair of adhesive sheets, at least a pair of threading components, at least a pull string, at least a pull component and pull string locking component. The skin wound closing apparatus provided in this invention includes a pair of adhesive sheets which are stick to the skin of the two sides of the wound, at least a pair of threading components positioned on the adhesive sheet and pull string which is set to thread between the threading cubes therefore the operator can close the wound simply by pulling the pull string, so it has the advantages of convenient operation and quick use. The skin wound closing apparatus also includes the pull components, so it can indicate the specific part of the pull string that the user needs to pull when closing the wound, which is convenient to use, thereby obtaining the best wound closure effect. The skin wound closing apparatus also includes a pull string locking component, so it can lock the part of the pull string pulled out by the pull component, so that the wound can be closed firmly.

### CROSS-REFERENCE TO RELATED APPLICATIONS

This application claims priority of four China patent applications, which are CN Patent Application No. 2020100685574, filed on Jan. 21, 2020, CN Patent Application No. 2020100685926, filed on Jan. 21, 2020, CN Patent Application No. 2020201432501, filed on Jan. 22, 2020, and CN Patent Application No. 2020201441939, filed on Jan. 22, 2020, the content of which is incorporated herein in its entirety by reference.

### FIELD

The present invention relates to a kind of skin wound closing apparatus, belonging to the field of medical equipment.

### BACKGROUND OF THE INVENTION

Traditionally, the skin wound closure methods are invasive, such as sutures with needle and thread or sutures with staples. Patients suffers not only the pain of suture but also the pain of the removal of the threads or staples after the wound is closed. The removal only causes secondary trauma but also leaves obvious scars. In recent years, a variety of noninvasive wound closure techniques have emerged, such as glue closure, zipper closure and tie closure. These techniques have the advantages of anesthesia free when are applied, causing no secondary trauma when the wound is closed, and leaving no obvious scars. However, these techniques have disadvantages such as cumbersome operations, time consuming, unstable closure and uneven vulnus juxtaposition, which unable to fully meet the clinical demands for fast operation, safety and beauty.

### SUMMARY OF THE INVENTION

In the view of the aforementioned issues, the present invention provides a suture-free skin wound closing apparatus that sutures wounds quickly, safely and neatly.

Many embodiments of the invention are related to a skin wound closing apparatus that can include a pair of adhesive sheets which are capable to stick on both sides of the wound respectively; at least a pair of threading components, each pair of the threading components symmetrically is positioned on the upper surface of the adhesive sheets respectively, and each the threading component is provided with threading holes; at least a pull strings, which symmetrically threads through all said threading holes in a manner that is symmetric to the center line of said pair of adhesive sheets, and alternately thread through the pair of adhesive sheets; at least a pull component, by which the operator pulls to drive the pair of adhesive sheets closer to each other so that the wound is closed; and pull string locking component, by which the unreeved string is locked. The unreeved string is the part of string pulled out by the pull string component when the pair of adhesive sheets are driven closer to each other to close the wound.

In many embodiments, the threading component is a threading board, which is provided with a plurality of threading holes.

In many embodiments, each the threading board is provided with threading perforation corresponding to and communicated with the threading holes, the threading perforation enables the pull string threading through the threading holes, the penetrating direction of the threading perforation is perpendicular to the length direction of the adhesive sheets.

In many embodiments, the threading components are threading cubes, each of which is provided with a threading hole.

In many embodiments, each of the adhesive sheets is arranged with a plurality of threading cubes which are lined up in a row along the wound, the threading cube is provided with the threading perforation communicated with the threading holes, which enable the pull string threading through the threading holes, the penetrating direction of the threading perforation is parallel to the length direction of the adhesive sheets.

In many embodiments, the pull string locking components are string locking squares, which are mounted on the adhesive sheets to compose a groove through which the unreeved pull string passes and is locked on the thread locking squares.

In many embodiments, the pull string locking component is a clamp style locker which is provided with a groove through which the unreeved pull string passes and is locked on the clamp style locker.

In many embodiments, the pull string locking component is a snap locker. The snap locker includes a snap locker base, which is mounted on the adhesive sheets, provided with a groove; and a button provided with tongue matching with the groove, the unreeved pull string passes through the groove and is compressed and locked in the groove by engaging the button with the snap locker base.

In many embodiments, the pull string locking component is a turn locker comprising including a turn locker base, mounted on the adhesive sheets and provided with a groove and further the groove provided with screw thread; a rotating component provided with a screw threaded rod of which the screw thread matches with the screw thread on the groove, the unreeved string passes through the groove, by engaging the rotating component with the turn locker base the unreeved string is locked in the groove.

In many embodiments, the pull string component is a spring locker including a shell mounted on the adhesive sheets; a spring arranged in the shell; a convex square, one end of which is connected with the spring and the other end extends out of the shell; a locking square, which is arranged on the same adhesive sheet as the shell, by engaging the locking square with the convex square, the pull string is locked.

In many embodiments, the pull string locking component is an elastic needle locker including an elastic needle mounting square, which is mounted on the adhesive sheets; a stopper provided with sliding groove; and an elastic needle, one end of which is positioned on the elastic needle mounting square, the other end of which cooperate with the block square to lock the unreeved string in the sliding groove, the elastic needle and the pulling string are at an obtuse angle when pulled by the operator.

In many embodiments, the pull string component is a spring-loaded locker including a fixing rod provided with penetrating thread locking perforation; a shell sleeving with the fixing rod and provided with a penetrating perforation for the pull string passing through and a fixing rod hole holding the fixing rod; and a spring positioned in the inner of the shell used for connecting the fixing rod and the shell. The direction of the thread locking perforation is the same as the direction of the penetrating perforation. When the spring is in the natural state, the thread locking perforation is not communicated with the penetrating perforation. At least when the spring is in a specific compressed state, the thread locking perforation is communicated with the penetrating perforation. When the unreeved thread passes both the thread locking perforation and the penetrating perforation, and the spring is in the natural state, the pull thread is locked by the spring-loaded locker.

In many embodiments, the spring-loaded locker including a first position limiter mounted on the fixing rod hole; and a second position limiter mounted on the outer side of the fixing rod in the shell, by the cooperation of the first position limiter and the second position limiter the fixing rod is prevented from falling out from the shell.

In many embodiments, the pull string component is an adhesive strip. The adhesive strip is used to lock the unreeved string on the adhesive sheets or skin.

In many embodiments, the number of the pull string is one, a pair of pull component are positioned on the two ends of the pull string respectively. The operator pulls the pull components in the direction parallel to the length direction of the adhesive sheets to drive the two adhesive sheets closer to each other, so that the wound is closed.

In many embodiments, the number of the pull string is two, two ends of the pull strings are positioned on the ends of the adhesive sheets respectively. The pull component is positioned at the middle of each of the adhesive sheets and positioned on the pull string, the operator pulls the pulling part at the opposite direction perpendicular to the extension of the wound to drive the two adhesive sheets closer to each other, so that the wound is closed.

In many embodiments, two sticklers were mounted on the two sides of the adhesive sheet, so that the adhesive sheets are stuck on the skin in a parallel manner.

In many embodiments, the pull components are a pull ring or an indicative part of the pull string.

In many embodiments, after the skin wound closing apparatus is stuck to the skin, and before the wound closing operation, the distance between the two adhesive sheets is 0.5 cm˜3.0 cm; after the wound closing operation, the distance between the two adhesive sheets is 0.2 cm˜2.0 cm.

In many embodiments, the adhesive sheets are arranged with a plurality of air permeable holes.

### THE ROLE AND EFFECT OF THE INVENTION

The invention related to a skin wound closing apparatus includes a pair of adhesive sheets which are stuck on both sides of the wound respectively; a plurality of threading cubes mounted on the adhesive sheets and pull string passing the threading cubes, so that the operator can close a wound by pulling the pull string. Accordingly, the skin wound closing apparatus has the advantages of convenient operation and quick use.

The invention related to a skin wound closing apparatus includes pull components, so that the apparatus indicates the operator the specific part of pull string that need to be pulled. Accordingly, the apparatus is convenient to use, thereby obtaining the best wound closure effect.

### BRIEF DESCRIPTION OF THE DRAWINGS

FIG. 1 is a schematic view showing the skin wound closing apparatus, according to the first embodiment of the present invention.

FIG. 2 is a top view showing the exemplary skin wound closing apparatus, according to the first embodiment of the present invention.

FIG. 3 is a schematic view showing the elastic needle string locker of the skin wound closing apparatus, according to the first embodiment of the present invention.

FIG. 4 is a schematic view showing the spring locker of the skin wound closing apparatus, according to the first embodiment of the present invention.

FIG. 5 shows the skin wound closing apparatus in use, according to the first embodiment of the present invention.

FIG. 6 is a schematic view showing the skin wound closing apparatus, according to the second embodiment of the present invention.

FIG. 7 is a top view showing the skin wound closing apparatus, according to the second embodiment of the present invention.

FIG. 8 shows the skin wound closing apparatus in use, according to the second embodiment of the present invention.

FIG. 9 shows the skin wound closing apparatus, according to the embodiment of the third present invention.

FIG. 10 is a top view showing the skin wound closing apparatus, according to the third embodiment of the present invention.

FIG. 11 shows the skin wound closing apparatus in use, according to the third embodiment of the present invention.

FIG. 12 is a schematic view showing the skin wound closing apparatus, according to the fourth embodiment of the present invention.

FIG. 13 is a top view showing the skin wound closing apparatus, according to the fourth embodiment of the present invention.

FIG. 14 is a schematic view showing the clamp style locker of the skin wound closing apparatus, according to the fourth embodiment of the present invention.

FIG. 15 is a schematic view showing the snap locker of the skin wound closing apparatus, according to the fourth embodiment of the present invention.

FIG. 16 is a schematic view showing the turn locker of the skin wound closing apparatus, according to the fourth embodiment of the present invention.

FIG. 17 shows the skin wound closing apparatus in use, according to the fourth embodiment of the present invention.

FIG. 18 is a schematic view showing the skin wound closing apparatus, according to the fifth embodiment of the present invention.

FIG. 19 is a top view showing the skin wound closing apparatus, according to the fifth embodiment of the present invention.

FIG. 20 shows the skin wound closing apparatus in use, according to the embodiment of the fifth present invention.

FIG. 21 shows the skin wound closing apparatus in use, according to the embodiment of the sixth present invention.

FIG. 22 is a top view showing the skin wound closing apparatus, according to the sixth embodiment of the present invention.

FIG. 23 shows the skin wound closing apparatus in use, according to the sixth embodiment of the present invention.

FIG. 24 is a schematic view showing the skin wound closing apparatus, according to the seventh embodiment of the present invention.

FIG. 25 is a top view showing the skin wound closing apparatus, according to the seventh embodiment of the present invention.

FIG. 26 is a schematic view showing the spring loaded locker of the skin wound closing apparatus, according to the seventh embodiment of the present invention;

FIG. 27 shows the skin wound closing apparatus in use, according to the seventh embodiment of the present invention.

FIG. 28 is a schematic view showing the skin wound closing apparatus, according to the eighth embodiment of the present invention.

FIG. 29 is a top view showing the skin wound closing apparatus, according to the eighth embodiment of the present invention.

FIG. 30 shows the skin wound closing apparatus in use, according to the eighth embodiment of the present invention.

### DETAILED DESCRIPTION

To make the arts, characteristic and purpose of the provided invention easy to understand, the following description details some examples and drawings of such apparatus was given.

### The First Embodiment

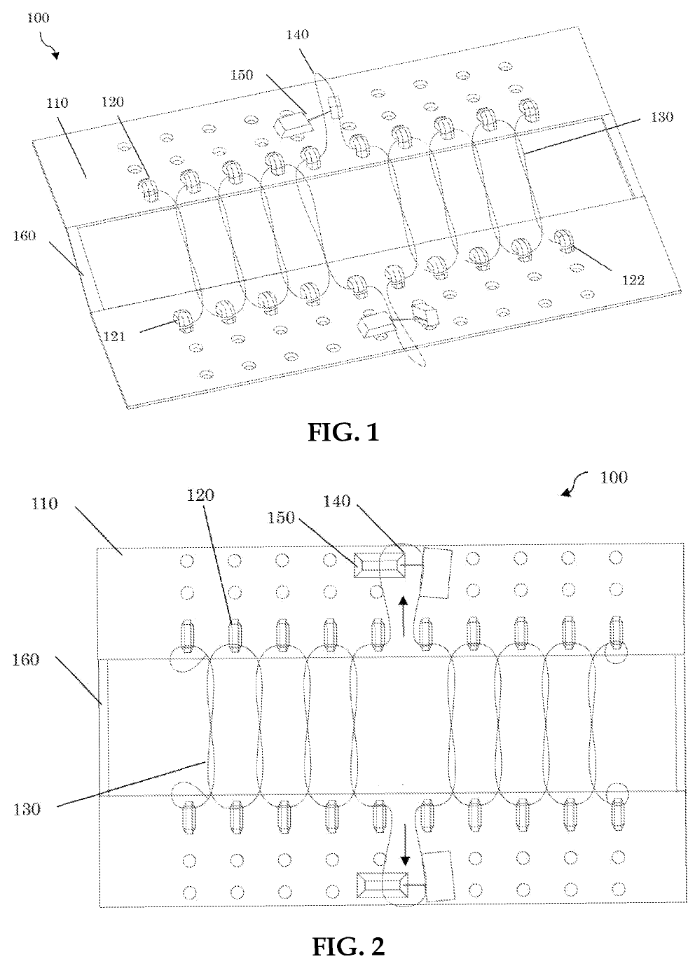

FIG. 1 and FIG. 2 respectively shows schematic and top views of the skin wound closing apparatus.

FIG. 1-2 shows the apparatus 100 provided in this embodiment includes a pair of adhesive sheets 110, a plurality pair of threading cubes 120, two pull strings 130, a pair of pull components 140, a pair of elastic needle lockers 150 and a pair of sticking strips 160.

The skin wound closing apparatus provided in this embodiment can be used in closing both human wounds and animal wounds. In this embodiment, the apparatus is used to close human wounds.

A pair of the adhesive sheets 110 is stuck to the two sides of the wound respectively and the extending length along the wound is 6 cm. The thickness of each adhesive sheet 110 is 3 mm. Each of the adhesive sheets is provided with a plurality of air permeable holes facilitating skin air exchange. More particularly, in this embodiment, the number of air permeable holes provided on each of the adhesive sheets 110 is 20, arranged in a 2×10 matrix.

In other embodiments, according to actual needs, the thickness of the adhesive sheet 110 is 1 mm˜4 mm and the extending length along the wound is 1 cm˜30 cm.

More particularly, when the skin wound closing apparatus 100 is used to close wound shorter than 1 cm, a 1 cm length adhesive sheet 110 is chosen; when the skin wound closing apparatus 100 is used to close wound of which length is 25 cm˜30 cm, a 30 cm length adhesive sheet is chosen.

In other embodiments, the adhesive sheet 110 side which is approaching to the other is provided with anti-bending board which is made of hard material. The skin closing apparatus provided with anti-bending board is generally used to close long and straight wound, preventing adhesive sheets 110 from distortion which has bad effect on wound closure.

Each pair of threading cubes 120 are symmetrically mounted the upper surface of adhesive sheets 110. All threading cubes 120 mounted on the same adhesive sheet 110 are line up in a row along the wound. More particularly, in this embodiment, the number of threading cubes 120 mounted the same adhesive sheets 110 is 10, lined up in a row along the wound and the distance between the neighboring two threading cubes mounted on the same adhesive sheet is 5 mm.

Each of the threading cubes 120 is arc shape provided a threading hole 121 and a threading perforation 122 communicated with the threading hole 121.

The threading perforation 122 facilitates the pull string 130 quickly threads through the threading hole 121, so that the skin wound closing apparatus 100 is quickly assembled and easy to produce.

The penetrating direction of the threading holes of the threading cubes 120 mounted on the same adhesive sheet 110 is parallel to the length direction of the adhesive sheet 110.

The pull string 130 is set to symmetrically thread through all the threading holes 121 with the center line of the pair of adhesive sheets 110 as symmetry axis and alternately thread through the pair of adhesive sheets 110. In other embodiments, the skin wound closing apparatus 100 may include a pull string 130, two and more pull strings 130. In this embodiment, the number of the pull strings 130 is two. In this embodiment, the pull strings 130 are evenly set between the pair of adhesive sheets 110. However, in cases of closing certain wounds (such as wound spanning joints), the pull strings 130 are set dense in the middle and sparse on the sides. The material of the pull string 730 is preferably PTFE or Parylene, the diameter of which is 0.2 mm˜1.0 mm. In this embodiment, the pull strings 730 are PTFE, the diameter of which is 0.5 mm.

FIG. 5 shows the skin wound closing apparatus 100 in use, according to the first embodiment of the present invention.

As shown in FIG. 5, for the convenience of description, the threading cubes 120 mounted on one of the adhesive sheets 110 are named threading cube A1 to threading cube A10 respectively and the threading cubes mounted on the other adhesive sheet 110 are named threading cube B1 to threading cube B10.

In this embodiment, the number of pull string 130 is two. One of the pull strings connects the two adhesive sheets 110 in a manner of threading through threading cube A1, threading cub B2, threading cub A3, threading cub B4, threading cube A5, threading cube A6, threading cube B7, threading cube A8, threading cube B9 and threading cube A10 successively. The two ends of the pull string 130 are tied to threading cube A1 and threading cube A10 respectively. The other pull string 130 connects the two adhesive sheets 110 in a manner of threading through threading cube B1, threading cub A2, threading cub B3, threading cub A4, threading cube B5, threading cube B6, threading cube A7, threading cube B8, threading cube A9 and threading cube B10 successively. The two ends of the pull string 130 are tied to threading cube B1 and threading cube B10.

A pair of pull components 140 is provided for operator pulling to drive adhesive sheets 110 closer to each other to close the wound. More particularly, the pull components 140 can be pull rings provided on pull strings 130 or indicative parts of pull strings such as colored parts, knotted parts and etc.

More particularly, in this embodiment, pull string components 140 are colored parts of pull string 130. Two pull components are positioned on where the pull string threads through threading cube A5 and threading cube A6 and where threads the pull string threads through threading cube B5 and threading cube B6 respectively.

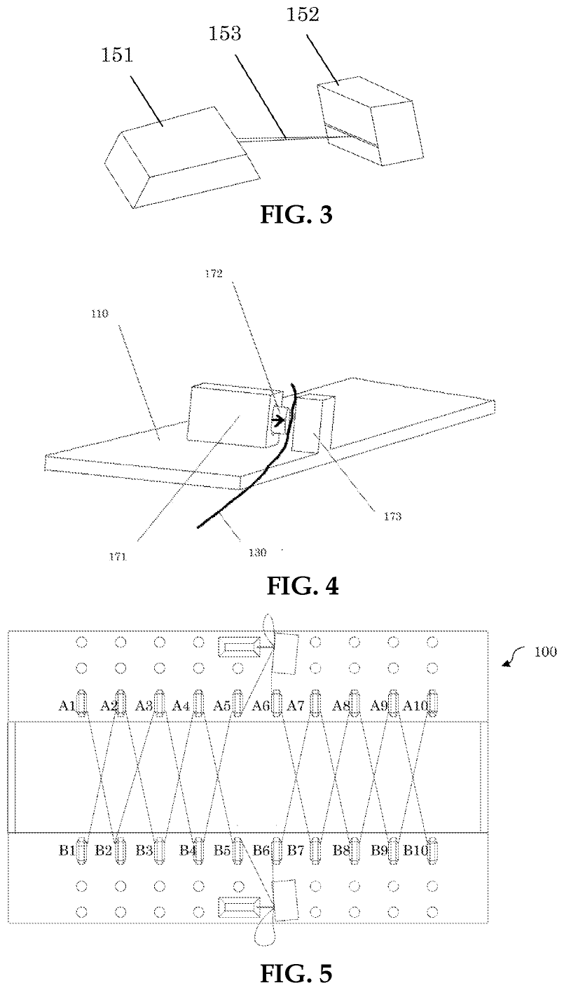

FIG. 3 is a schematic view showing the elastic needle string locker of the skin wound closing apparatus, according to the first embodiment of the present invention.

As shown in FIG. 3, a pair of elastic needle lockers 150 is positioned on the upper surface of adhesive sheets 110 and the corresponding position of pull components 140 to lock the unreeved pull string. The unreeved pull string is the part of pull string pulled out when adhesive sheets are driver closer to each other. The unreeved pull string can be locked on elastic needle lockers 150 in advance and can also be locked on elastic needle lockers 150 after the wound is closed. In this embodiment, the operator locks the unreeved pull string after the wound is closed.

Elastic needle locker 150 includes an elastic needle fixing square 150, a stopper 152 and an elastic needle 153.

The elastic needle fixing square 151 is mounted on the adhesive sheet 110.

The block square 152 is provided with sliding groove which enables pull string 110 passing through.

One end of the elastic needle 153 is positioned on the elastic needle fixing square 151, and the other end of elastic needle 153 engages with the stopper 151 to lock the unreeved string in the sliding groove. The elastic needle 153 and the pull string 130 are at an obtuse angle when pulled by the operator, so that the pull strings 130 can only move along the direction of pulling and cannot move along the opposite direction. The elastic needle 153 is resilient and can be repeatedly toggled. The elastic needle 153 also has good elasticity and is able to lock the unreeved pull line in the sliding groove.

In other embodiments, the elastic needle locker 150 can be replaced by a spring locker 170.

FIG. 4 is a schematic view showing the spring locker of the skin wound closing apparatus, according to the first embodiment of the present invention.

As shown in FIG. 4, the spring locker 170 includes a shell 171, a spring (not shown in the FIG), a convex square 172,and a locking square 173.

The shell 171 is positioned on the adhesive sheet 110.

The spring is positioned in the shell 171.

One end of the convex square 172 is connected with the spring and the other end of which extends out of the shell 171.

The locking square 173 is positioned on the same adhesive sheet 110 as the shell 171 positioned. which is arranged on the same adhesive sheet as the shell. By engaging the locking square with the convex square, the pull string is locked.

A pair of sticking strips 160 is stuck to the two sides of the adhesive sheet 110 respectively so that the adhesive sheets 110 are mounted on skin in a parallel manner. The length and width of the sticking strips 160 are set according to actual demands. The sticking strips 160 are made of soft materials and more particularly, in this embodiment, the sticking strips 160 are soft strips.

The instruction of the embodiment of the skin wound closing apparatus 100 is shown as below:

Position the adhesive sheets 110 at two sides of the wound respectively, and the distance between the adhesive sheets 110 is 0.5 cm˜25 cm. After the wound is closed, the distance between the adhesive sheets 110 is 0.2 cm˜2.0 cm, and the distance depends on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull components 140 in the direction of the arrow showed in FIG. 2 to drive the adhesive sheets 110 closer to each other to close the wound. At the moment, the distance between two adhesive sheets 110 is 0.2 cm˜2.0 cm. After the wound is closed, the operator locks the part of pull string of the pull component 140 with the elastic needle locker 150.

In the process of the wound healing, the operator can adjust the position of the pull string 130 locked by the elastic needle locker 150 to adjust the tightness of the pull string 130. More particularly, if the pull string 130 is loose, pull the pull component 140 until the tightness is suitable; if the pull string 130 is too tight, the operator can pull the elastic needle 153 aside, adjust the pull string 130 to suitable position and release the elastic needle 153 to lock the unreeved pull string.

After the wound is healed, it is not necessary to remove the pull string 130 and the operator can separate the adhesive sheets 110 from skin directly.

The Function and Effect of the Embodiment

According to the first embodiment of the skin wound closing apparatus, the skin wound closing apparatus includes a pair of adhesive sheets stuck to the two side of the wound respectively, a plurality of threading cubes mounted on the adhesive sheets, and pull string alternately threading through the threading cubes, therefore, the operator can close the wound only by pull the pull string. Consequentially, this invention enjoys the benefits of convenient operation and easy to use.

According to the first embodiment of the skin wound closing apparatus, the skin wound closing apparatus includes the pull components, therefore, the skin wound closing apparatus can indicate to the operator the specific part of the pull string that is used for pulling. Consequentially, this invention is easy to use and the best closure of the wound can be realize.

According to the first embodiment of the skin wound closing apparatus, the skin wound closing apparatus includes the elastic lockers therefore the skin wound closing apparatus can lock the unreeved pull string. Consequentially, a stable wound closure is realized.

Furthermore, according to the first embodiment of the skin wound closing apparatus, the skin wound closing apparatus adopts two pull strings and in the middle of the pull string is provided with a pull component therefore the operator can symmetrically pull the pull string so that the wound is closed neatly.

### The Second Embodiment

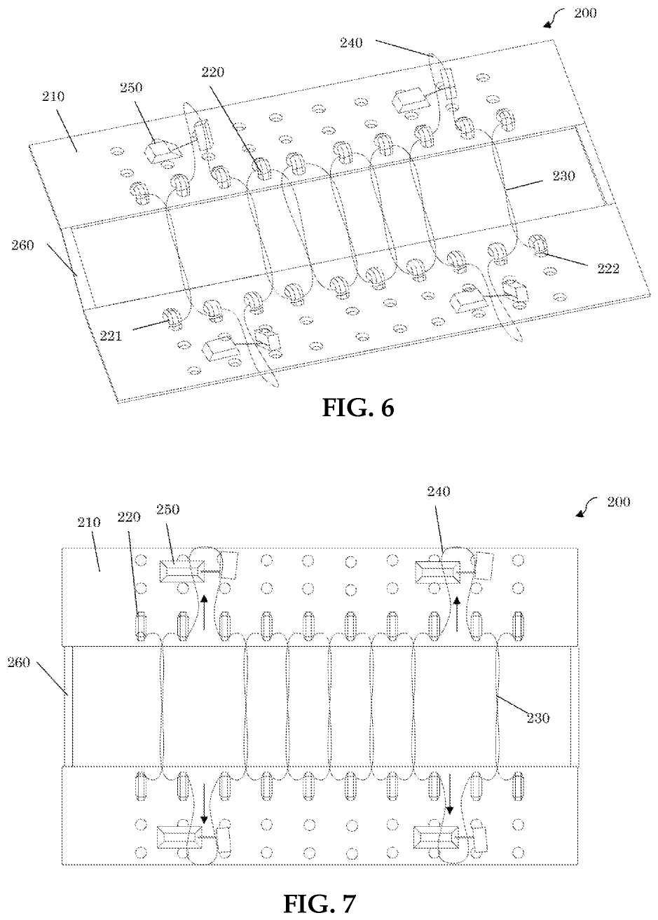

FIG. 6 and FIG. 7 respectively shows schematic and top views of the skin wound closing apparatus. FIG. 6-7 show the apparatus 200 provided in this embodiment includes a pair of adhesive sheets 210, a plurality pair of threading 220, a pull string 230, a pair of pull part 240, a pair of elastic needle locker 250 and a pair of sticklers 260.

A pair of the adhesive sheets 210 is stuck to the two sides of the wound respectively and the extending length along the wound is 15 cm. The thickness of each adhesive sheet 210 is 3 mm. Each of the adhesive sheets 210 is provided with a plurality of air permeable holes facilitating patient's skin air exchange. More particularly, in this embodiment, the number of air permeable holes provided on each of the adhesive sheets 210 is 20, and are arranged in a 2×10 matrix.

In other embodiments, according to actual needs, the thickness of the adhesive sheet is 1 mm˜4 mm and the extending length along the wound is 1 cm˜30 cm.

More particularly, when the skin wound closing apparatus is used to close wound shorter than 1 cm, a 1 cm length adhesive sheet 210 is chosen; when the skin wound closing apparatus is used to close wound of which length is 20 cm˜30 cm, a 30 cm length adhesive sheet is chosen.

In other embodiments, the adhesive sheet 210 side which is approaching to the other is provided with anti-bending board which is made of hard material. The skin closing apparatus 200 provided with anti-bending board is generally used to close long and straight wounds, preventing adhesive sheets 210 from distortion which has bad effect on wound closure.

Each pair of threading cubes 220 are symmetrically mounted the upper surface of adhesive sheets 210. All threading cubes 220 mounted on the same adhesive sheet 210 are line up in a row along the wound. In other embodiments, the plurality of threading cubes can line up in other manners. The distance of the neighboring threading cubes 220 mounted on the same adhesive sheets 210 is 3 mm˜10 mm. More particularly, in this embodiment, the number of threading cubes 220 mounted on each of adhesive sheets 210 is 10, and the distance between neighboring two threading cubes mounted on the same adhesive sheet is 10 mm.

Each of the threading cubes 220 is arc shape provided a threading hole 221 and a threading perforation 222 communicated with the threading hole 221.

The penetrating direction of the threading perforation 210 of the threading cubes 220 mounted on the same adhesive sheet 210 is parallel to the length direction of the adhesive sheet 210.

The pull string 230 is set to symmetrically thread through all the threading holes 221 with the center line of the pair of adhesive sheets 210 as symmetry axis and alternately threads through the pair of adhesive sheets 210. In other embodiments, the skin wound closing apparatus may include a pull string 230, two or more pull string 230 depending on the manner of pulling. In this embodiment, the number of pull strings 230 is two. Generally, the pull strings 230 are evenly set between the pair of adhesive sheets 210. However, in the cases closing certain wounds(such as wound spanning joints), the pull strings 230 are set dense in the middle and sparse on the sides. The material of the pull string is preferably PTFE or Parylene, the diameter of which is 0.2 mm˜1.0 mm. In this embodiment, the pull strings are PTFE, the diameter of which is 0.5 mm.

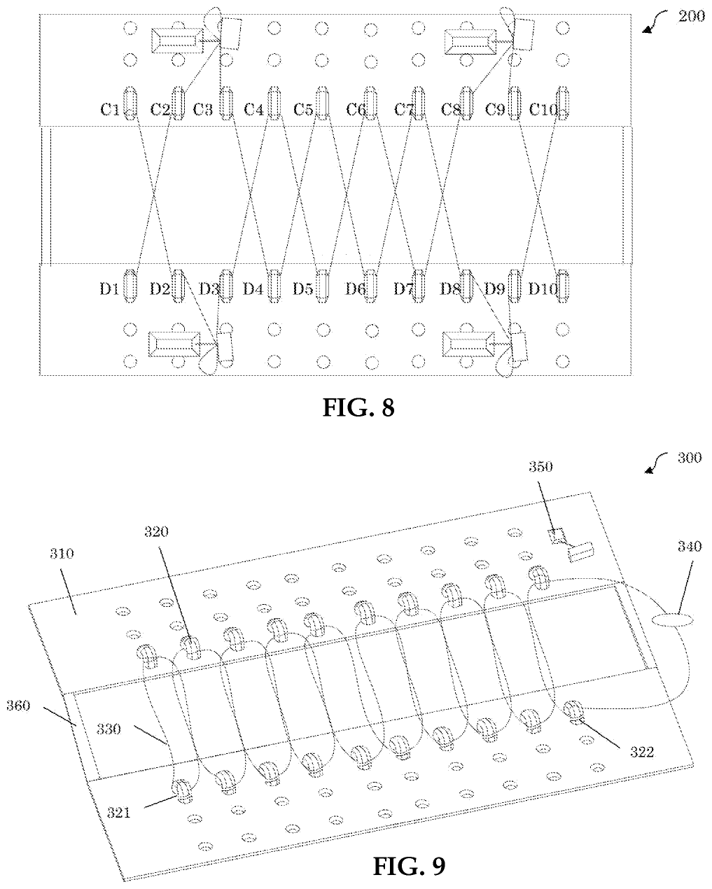

FIG. 8 shows the skin wound closing apparatus in use, according to the second embodiment of the present invention.

As shown in FIG. 8, for the convenience of description, the threading cubes 220 mounted on one of the adhesive sheets 210 are named threading cube C1 to threading cube C10 respectively and the threading cubes mounted on the other adhesive sheet are named threading cube D1 to threading cube D10.

In this embodiment, the number of pull string 230 is two. One of the pull strings 230 connects the two adhesive sheets 210 in a manner of threading through threading cube C1, threading cub D2, threading cub D3, threading cub C4, threading cube D5, threading cube C6, threading cube D7, threading cube C8, threading cube C9 and threading cube D10 successively. The two ends of the pull string 230 are tied to threading cube C1 and threading cube D10 respectively. The other pull string 230 connects the two adhesive sheets 210 in a manner of threading through threading cube D1, threading cub C2, threading cub C3, threading cub D4, threading cube C5, threading cube D6, threading cube C7, threading cube D8, threading cube D9 and threading cube C10 successively. The two ends of the pull string 230 are tied to threading cube D1 and threading cube C10.

A pair of pull components 240 is provided for operator pulling to drive adhesive sheets 210 closer to each other to close the wound. More particularly, pull components 240 can be pull rings provided on pull strings 230 or indicative parts of pull strings 230 such as a colored part, knotted parts and etc.

More particularly, in this embodiment, the pull string components 240 are colored parts of pull string 230. Four pull components are positioned on the certain points of the pull string 230 which are between threading cube D2 and D3, between threading cube C2 and C3, between threading cube D8 and D9, between threading cube C8 and C8 respectively.

Two pair of elastic needle lockers 250 are positioned on the upper surface of adhesive sheets 210 and the corresponding position of pull components 140 to lock the unreeved pull string. The unreeved pull string is the part of pull string pulled out by the pull part when adhesive sheets 210 are driven closer to each other to close the wound. The unreeved pull string can be locked on elastic needle lockers 250 in advance and can also be locked on elastic needle lockers 250 after the wound is closed. In this embodiment, the operator locks the unreeved pull string after the wound is closed.

The structure of the elastic needle locker is exactly the same as the structure showed in the first embodiment and is not detailed here.

In other embodiments, the elastic needle locker 250 can be replaced by a spring locker 270.

FIG. 4 is a schematic view showing the spring locker of the skin wound closing apparatus, according to the first embodiment of the present invention.

The structure of the elastic needle locker is exactly the same as the structure showed in the first embodiment and is not detailed here.

A pair of sticking strips 260 is stuck to the two sides of the adhesive sheet 210 respectively so that the adhesive sheets 210 are mounted on skin in a parallel manner. The length and width of the sticking strips 260 are set according to actual demands. The sticking strips 260 are made of soft materials and more particularly, in this embodiment, the sticking strips 260 are soft strips.

The instruction of the embodiment of the skin wound closing apparatus 200 is shown as below:

Position the adhesive sheets 210 at two sides of the wound respectively, and the distance between the adhesive sheets 210 is 0.5 cm˜25 cm. After the wound is closed, the distance between the adhesive sheets 210 is 0.2 cm˜2.0 cm, depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull components 240 in the direction of the arrow showed in FIG. 7 to drive the adhesive sheets 210 closer to each other to close the wound. After the wound is closed, the operator locks the part of pull string of the pull component 240 with the elastic needle locker 250.

In the process of the wound healing, the operator can adjust the position of the pull string 230 locked by the elastic needle locker 250 and adjust the tightness of pull string 230. More particularly, if the pull string is loose, pull the pull component 240 until the tightness is suitable; if the pull string 230 is too tight, the operator can pull the elastic needle 253 aside, adjust the pull string 230 to suitable position and release the elastic needle 253 to lock the unreeved pull string.

After the wound is healed, it is not necessary to remove the pull string 230 and the operator can separate the adhesive sheets 210 from skin directly.

The Function and Effect of the Embodiment

According to the second embodiment of the skin wound closing apparatus, the skin wound closing apparatus enjoys the benefits mentioned in the first embodiment. Moreover, the second skin wound closing apparatus includes two pull strings and each of which is provided with two pull component positioned on the top third and bottom third therefore the operator can use four pull components at the same time. Consequentially, larger type of adhesive sheets can be driven closer to each other. Compared to the first embodiment, this embodiment of the skin wound closing apparatus is more suitable to close long wounds.

### The Third Embodiment

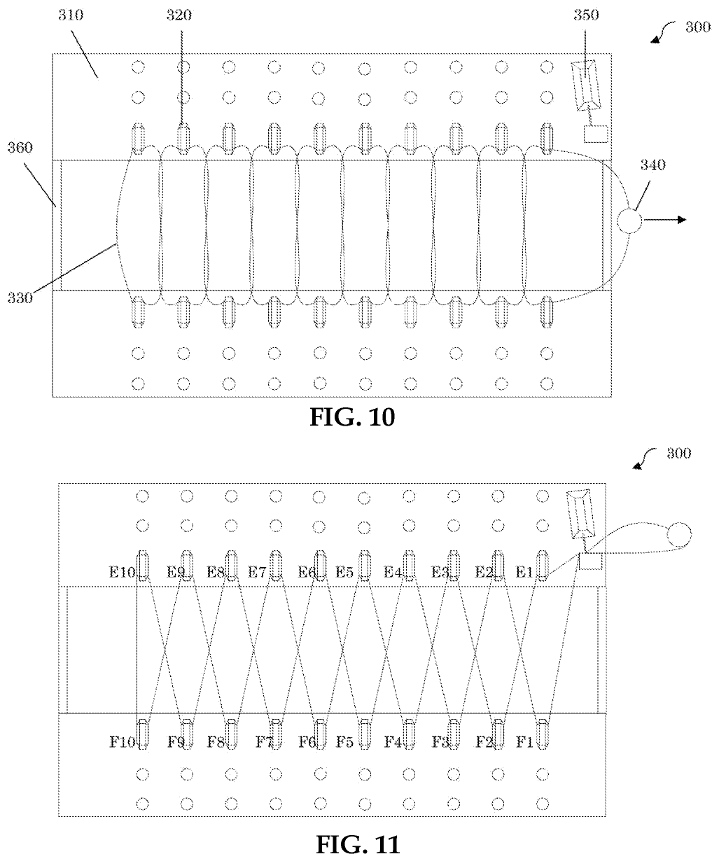

FIG. 9 and FIG. 10 respectively shows schematic and top views of the skin wound closing apparatus.

FIG. 9-10 show the apparatus 300 provided in this embodiment includes a pair of adhesive sheets 310, a plurality pair of threading 320, a pull string 330, a pair of pull parts 340, a pair of elastic needle locker 350 and a pair of sticklers 360.

A pair of the adhesive sheets 310 is stuck to the two sides of the wound respectively and the extending length along the wound is 6 cm. The thickness of each adhesive sheet 310 is 3 mm. Each of the adhesive sheets 310 is provided with a plurality of air permeable holes facilitating patient's skin air exchange. More particularly, in this embodiment, the number of air permeable holes provided on each of the adhesive sheets 310 is 20, and are arranged in a 2×10 matrix.

In other embodiments, according to actual needs, the thickness of the adhesive sheet 310 is 1 mm˜4 mm and the extending length along the wound is 1 cm˜30 cm.

More particularly, when the skin wound closing apparatus 300 is used to close wound shorter than 1 cm, a 1 cm length adhesive sheet 310 is chosen; when the skin wound closing apparatus is used to close wound of which length is 25 cm˜30 cm, a 30 cm length adhesive sheet 310 is chosen.

In other embodiments, the adhesive sheet 310 side which is approaching to the other is provided with anti-bending board which is made of hard material. The skin closing apparatus 300 provided with anti-bending board is generally used to close long and straight wounds, preventing adhesive sheets 310 from distortion which has bad effect on wound closure.

Each pair of threading cubes 320 are symmetrically mounted the upper surface of adhesive sheets 310. All threading cubes 320 mounted on the same adhesive sheet 310 are line up in a row along the wound. In other embodiments, the plurality of threading cubes 320 can line up in other manners. The distance of the neighboring threading cubes 320 mounted on the same adhesive sheets 310 is 3 mm˜10 mm. More particularly, in this embodiment, the number of threading cubes 320 mounted on each of adhesive sheets 310 is 10, and the distance between neighboring two threading cubes mounted on the same adhesive sheet is 5 mm.

Each of the threading cubes 320 is arc shape provided with a threading hole 321 and a threading perforation 322 communicated with the threading hole 321.

The threading perforation 322 facilitates the pull string 330 passing through the threading holes 321 therefore the skin wound closing apparatus 300 can be assembled in a short time and easy to produce.

The penetrating direction of the threading perforation 310 of the threading cubes 320 mounted on the same adhesive sheet 310 is parallel to the length direction of the adhesive sheet 310.

The pull string 330 is set to symmetrically thread through all the threading holes 321 with the center line of the pair of adhesive sheets 310 as symmetry axis and alternately threads through the pair of adhesive sheets 310. In other embodiments, the skin wound closing apparatus may include a pull string 330, two or more pull string 330 depending on the manner of pulling. In this embodiment, the number of pull string 230 is one. Generally, the pull strings 330 are evenly set between the pair of adhesive sheets 310. However, in the cases closing certain wounds(such as wound spanning joints), the pull strings 330 are set dense in the middle and sparse on the sides. The material of the pull string 330 is preferably PTFE or Parylene, the diameter of which is 0.2 mm˜1.0 mm. In this embodiment, the pull strings are PTFE, the diameter of which is 0.5 mm.

FIG. 11 shows the skin wound closing apparatus in use, according to the third embodiment of the present invention.

As shown in FIG. 11, for the convenience of description, the threading cubes 320 mounted on one of the adhesive sheets 310 are named threading cube E1 to threading cube E10 respectively and the threading cubes mounted on the other adhesive sheet are named threading cube F1 to threading cube F10.

In this embodiment, the number of pull string 330 is one. The pull string 330 connects the two adhesive sheets 310 by threading through threading cube E1, threading cub F2, threading cub E3, threading cub F4, threading cube E5, threading cube F6, threading cube E7, threading cube F8, threading cube E9, threading cube F10, threading cube E10, threading cub F9, threading cub E8, threading cub F7, threading cube E6, threading cube F5, threading cube E4, threading cube F3, threading cube E2 and threading cube F1 successively. The two ends of the pull string 330 are tied to a pull component 340, forming a closed loop.

A pull component 340 is provided for operator pulling to drive adhesive sheets 310 closer to each other to close the wound. More particularly, pull component 340 can be a pull rings provided on pull strings 330 or an indicative part of the pull string 330 such as a colored part, knotted parts and etc. More particularly, in this embodiment, the pull string component 340 is a pull ring. The two ends of the pull string 330 are tied on the pull ring 340.

A pair of elastic needle lockers 350 is positioned on the upper surface of adhesive sheets 310 and the corresponding position of pull component 340 to lock the unreeved pull string. The unreeved pull string is the part of pull string pulled out by the pull part when adhesive sheets 310 are driven closer to each other to close the wound. The unreeved pull string can be locked on elastic needle lockers 350 in advance and can also be locked on elastic needle lockers 350 after the wound is closed. In this embodiment, the operator locks the unreeved pull string after the wound is closed.

The structure of the elastic needle locker is exactly the same as the structure showed in the first embodiment and is not detailed here.

The structure of the elastic needle locker 350 is exactly the same as the structure showed in the first embodiment and is not detailed here.

In other embodiments, the elastic needle locker 350 can be replaced by a spring locker 370. The structure of the spring locker 370 is exactly the same as the structure showed in the first embodiment and is not detailed here.

A pair of sticking strips 360 is stuck to the two sides of the adhesive sheet 310 respectively so that the adhesive sheets 310 are mounted on skin in a parallel manner. The length and width of the sticking strips 360 are set according to actual demands. The sticking strips 360 are made of soft materials and more particularly, in this embodiment, the sticking strips 360 are soft strips.

The instruction of the embodiment of the skin wound closing apparatus 300 is shown as below:

Position the adhesive sheets 310 at two sides of the wound respectively, and the distance between the adhesive sheets 310 is 0.5 cm˜25 cm. After the wound is closed, the distance between the adhesive sheets 310 is 0.2 cm˜2.0 cm, depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull component 340 in the direction of the arrow showed in FIG. 10 to drive the adhesive sheets 310 closer to each other to close the wound. After the wound is closed, the operator locks the unreeved pull string pulled out by the pull component 340 with the elastic needle locker 350.

In the process of the wound healing, the operator can adjust the position of the pull string 330 locked by the elastic needle locker 350 to adjust the tightness of pull string 330. More particularly, if the pull string is loose, pull the pull component 340 until the tightness is suitable; if the pull string 330 is too tight, the operator can pull the elastic needle 353 aside, adjust the pull string 330 to suitable position and release the elastic needle 353 to lock the unreeved pull string.

After the wound is healed, it is not necessary to remove the pull string 330 and the operator can separate the adhesive sheets 310 from skin directly.

The Function and Effect of the Embodiment

According to the third embodiment of the skin wound closing apparatus, the skin wound closing apparatus enjoys the benefits mentioned in the first embodiment. Moreover, the third skin wound closing apparatus includes one pull string and the two ends of which are connected to the pull ring therefore the operator can use the skin wound closing apparatus with one hand to drive the adhesive sheets closer to each other to close the wound. Compared to the skin wound closing apparatus provided in the first embodiment, the skin wound closing apparatus provided in this embodiment is more suitable single use, especially suitable for individual personal self-rescue in field, on the battlefield and in similar situations.

### The Fourth Embodiment

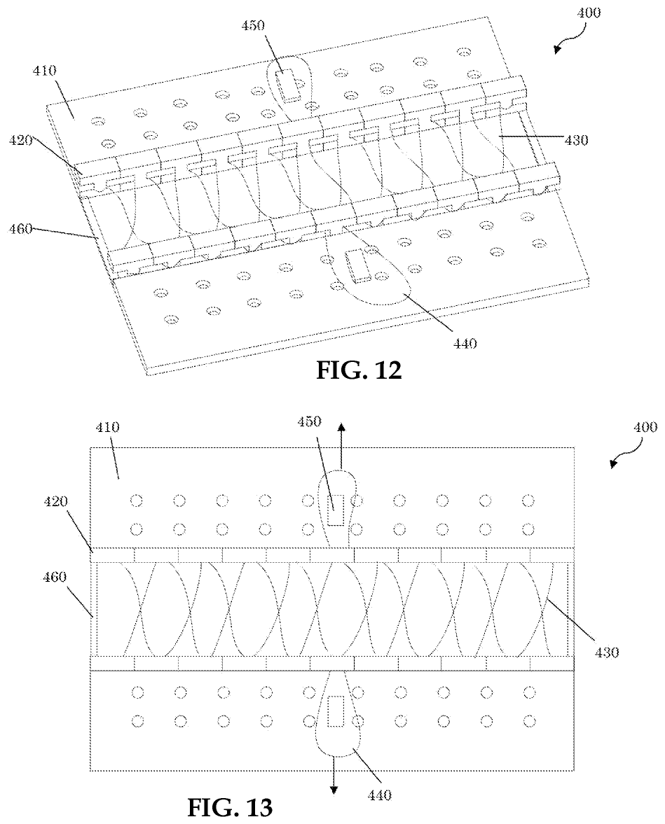

FIG. 12 and FIG. 13 respectively show schematic and top views of the skin wound closing apparatus.

FIG. 12-13 shows the apparatus 400 provided in this embodiment includes a pair of adhesive sheets 410, a pair of threading boards 420, two pull strings 430, a pair of pull parts 440, a pair of thread locking squares 450 and a pair of sticking strips 460.

The skin wound closing apparatus 400 provided in this embodiment can be used in closing both human wounds and animal wounds. In this embodiment, the skin wound closing apparatus 400 is used to close human wound.

A pair of the adhesive sheets 410 is stuck to the two sides of the wound respectively and the extending length along the wound is 6 cm. The thickness of each adhesive sheet 410 is 3 mm. Each of the adhesive sheets 410 is provided with a plurality of air permeable holes facilitating patient's skin air exchange. More particularly, in this embodiment, the number of air permeable holes provided on each of the adhesive sheets 410 is 20, and are arranged in a 2×10 matrix.

In other embodiments, according to actual needs, the thickness of the adhesive sheet 410 is 1 mm˜4 mm and the extending length along the wound is 1 cm˜30 cm.

More particularly, when the skin wound closing apparatus 400 is used to close wound shorter than 1 cm, a 1 cm length adhesive sheet 410 is chosen; when the skin wound closing apparatus is used to close wound of which length is 25 cm˜30 cm, a 30 cm length adhesive sheet 410 is chosen.

In other embodiments, the adhesive sheet 410 side which is approaching to the other is provided with anti-bending board which is made of hard material. The skin closing apparatus 400 provided with anti-bending board is generally used to close long and straight wounds, preventing adhesive sheets 410 from distortion which has bad effect on wound closure.

The pair of threading boards 420 are symmetrically mounted the upper surface of adhesive sheets 310. Each of the threading boards 420 is provided with a plurality of threading holes 421. More particularly, in this embodiment, each of the threading boards 420 is provided with 10 threading holes 421 and threading perforations 422 corresponding to and communicated with the threading holes 421. The threading perforations 422 facilitate the pull string thread through threading holes 421 therefore the skin wound closing apparatus can be assembled in a short time and easy to produce.

The penetrating direction of the threading holes 420 is perpendicular to the length direction of the adhesive sheets 410.

The pull string 430 is set to symmetrically thread through all the threading holes 421 with the center line of the pair of adhesive sheets 410 as symmetry axis and alternately threads through the pair of adhesive sheets 410. In other embodiments, the skin wound closing apparatus may include a pull string 430, two or more pull string 430 depending on the manner of pulling. In this embodiment, the number of pull strings 430 is two. In this embodiment, the pull string 430 is evenly set between the pair of adhesive sheets 410. However, in the cases of closing certain wounds (such as wound spanning joints), the pull string 430 is set dense in the middle and sparse on the sides. The material of the pull string 430 is preferably PTFE or Parylene, the diameter of which is 0.2 mm˜1.0 mm. In this embodiment, the pull string is PTFE, the diameter of which is 0.5 mm.

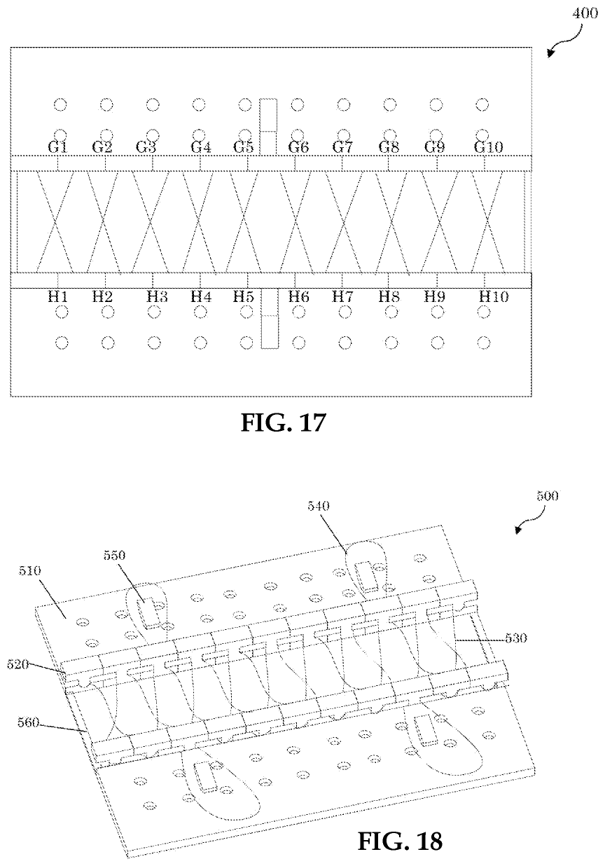

FIG. 17 shows the skin wound closing apparatus in use, according to the fourth embodiment of the present invention.

As shown in FIG. 17, for the convenience of description, the threading holes 421 mounted on one of the threading boards 420 are named threading hole G1 to threading hole G10 respectively and the threading holes 421 mounted on the other threading board 420 are named threading hole H1 to threading hole H10.

In this embodiment, the number of pull strings 430 is two. One of the pull strings 430 connects the two adhesive sheets 410 by threading through threading hole G1, threading cub H1, threading hole H2, threading hole G2, threading hole G3, threading hole H3, threading hole H4, threading hole G4, threading hole G5, threading hole H5, threading hole H6, threading hole G6, threading hole G7, threading hole H7, threading hole H8, threading hole G8, threading hole G9, threading hole H10 and threading hole G10 successively. The two ends of the pull string 430 are tied to the threading board 420. The other pull string 430 connects the two adhesive sheets 410 by threading through threading hole H1, threading cub G1, threading hole G2, threading hole H2, threading hole H3, threading hole G3, threading hole G4, threading hole H4, threading hole H5, threading hole G5, threading hole G6, threading hole H6, threading hole H7, threading hole G7, threading hole G8, threading hole H8, threading hole H9, threading hole G10 and threading hole H10 successively. The two ends of the pull string 430 are tied to the threading board 420.

A pull component 440 is provided for the operator pulling to drive adhesive sheets 410 closer to each other to close the wound.

More particularly, the pull component 440 can be a pull ring provided on pull strings 430 or an indicative part of the pull string 430 such as a colored part, knotted part and etc.

More particularly, in this embodiment, the pull string components 440 are colored parts of the pull string 430. The two pull components are positioned on the part of the pull string 430 where threads through threading hole G5 and threading hole G6 and where the pull string 430 threads through threading hole H5 and threading hole H6 respectively.

The string locking square 450 is positioned on the upper surface of adhesive sheets 410 and the corresponding position of pull component 440, forming a groove through which the unreeved string passes and by which is locked.

The string locking square 450 can be any shape. In this embodiment, a wedge string locking square is chosen. In other embodiments, in order to enhance the locking strength glue can be coated on the surface of string locking square 450 to lock the unreeved pull string.

In other embodiments, the string locking square 450 can be replaced by any one of clam style locker 470, snap locker 480 or turn locker 490.

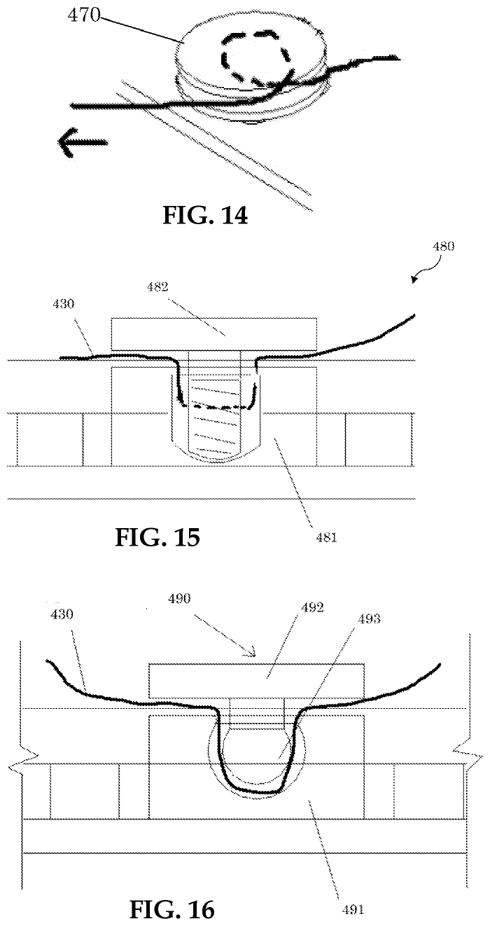

FIG. 14 shows the schematic view showing the clamp style locker provided in the fourth embodiment of the skin wound closing apparatus.

As shown in FIG. 14, the clam style locker 470 is provided with groove facilitating the pull string 430 passing through. The unreeved pull string is wound around the groove and is locked by the clap style locker 470.

FIG. 15 shows the schematic view showing the snap locker provided in the fourth embodiment of the skin wound closing apparatus.

As shown in FIG. 15, the snap locker 480 includes a snap locker base 481 and a button 482. The snap locker base is mounted on the adhesive sheets 410 and provided with a groove. The button 482 is provided with a tongue matching with the groove. The unreeved string passes through the groove. By engaging the button 482 and the snap locker base the unreeved string is locked.

FIG. 16 shows the schematic view showing the turn locker provided in the fourth embodiment of the skin wound closing apparatus.

As shown in FIG. 16, the turn locker 490 includes a turn locker base 491 and rotating component 492. The turn locker base 491 was mounted on the adhesive sheet 410 and provided with a groove. Furthermore, the groove is provided with screw threads. The rotating component is provided with screw threaded rod 493 matching with the screw threads in the groove. The unreeved string passes through the groove and is locked in the groove by engaging the rotating component 492 and turn locker base 491.

A pair of sticking strips 460 is stuck to the two sides of the adhesive sheet 410 respectively so that the adhesive sheets 410 are mounted on skin in a parallel manner. The length and width of the sticking strips 460 are set according to actual demands. The sticking strips 460 are made of soft materials and more particularly, in this embodiment, the sticking strips 460 are soft strips.

The instruction of the embodiment of the skin wound closing apparatus 400 is shown as below:

Position the adhesive sheets 410 at two sides of the wound respectively, and the distance between the adhesive sheets 410 is 0.5 cm˜25 cm. After the wound is closed, the distance between the adhesive sheets 410 is 0.2 cm˜2.0 cm, the specific distance depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull component 440 in the direction of the arrow showed in FIG. 2 to drive the adhesive sheets 410 closer to each other to close the wound. After the wound is closed, the operator locks the unreeved pull string pulled out by the pull component 440 with the string locking squares 450.

In the process of the wound healing, the operator can adjust the position of the pull string 430 locked by the string locking squares 450 to adjust the tightness of the pull string 430. More particularly, if the pull string is loose, pull out more pull string 430 until the tightness is suitable; if the pull string 430 is too tight, the operator can loosen a certain length of the pull string 430 from the string locking square 550.

After the wound is healed, it is not necessary to remove the pull string 430 and the operator can separate the adhesive sheets 310 from skin directly.

The Function and Effect of the Embodiment

According to the fourth embodiment of the skin wound closing apparatus, the skin wound closing apparatus enjoys the benefits mentioned in the first embodiment. Moreover, the fourth skin wound closing apparatus includes the string locking squares therefore the skin wound closing apparatus can lock the unreeved part pulled out by the pull component. Consequentially, the wound is closed more stably.

Furthermore, the skin wound closing apparatus provide in the fourth embodiment adopts 2 pull strings a pull component is positioned at the middle of the pull string. Consequentially, the operator can pull the pull string in a symmetrical manner so that the wound can close neatly.

### The Fifth Embodiment

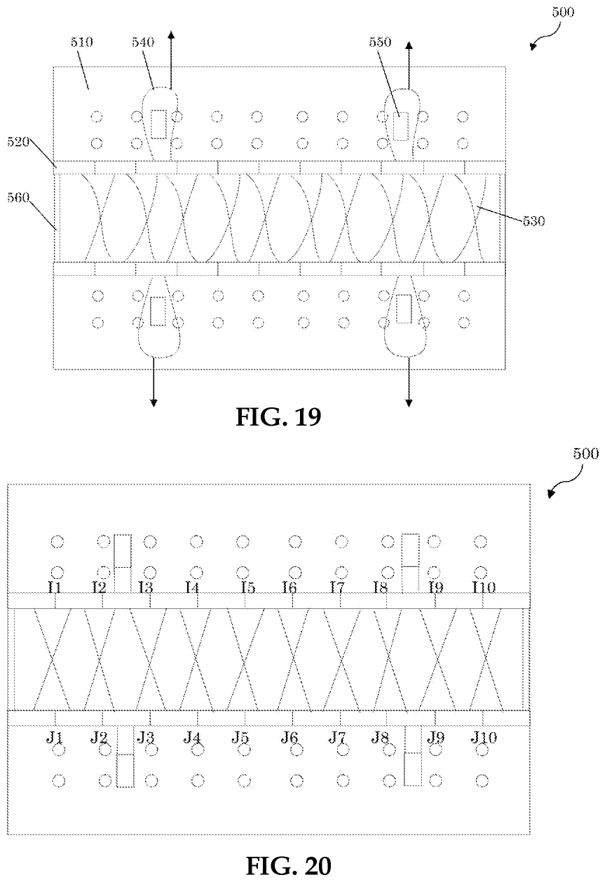

FIG. 18 and FIG. 19 respectively show schematic and top views of the skin wound closing apparatus according to the fifth embodiment of the skin wound closing apparatus.

FIG. 18-19 shows the apparatus 500 provided in this embodiment includes a pair of adhesive sheets 510, a pair of threading boards 520, a pull string 530, a pair of pull parts 540, a pair of thread locking squares 550 and a pair of sticking strips 560.

The skin wound closing apparatus 500 provided in this embodiment can be used in closing both human wounds and animal wounds. In this embodiment, the skin wound closing apparatus 500 is used to close human wounds.

A pair of the adhesive sheets 510 is stuck to the two sides of the wound respectively and the extending length along the wound is 6 cm. The thickness of each adhesive sheet 510 is 3 mm. Each of the adhesive sheets 510 is provided with a plurality of air permeable holes facilitating patient's skin air exchange. More particularly, in this embodiment, the number of air permeable holes provided on each of the adhesive sheets 510 is 20, and are arranged in a 2×10 matrix.

In other embodiments, according to actual needs, the thickness of the adhesive sheet 510 is 1 mm˜4 mm and the extending length along the wound is 1 cm˜30 cm.

More particularly, when the skin wound closing apparatus 500 is used to close wound shorter than 1 cm, a 1 cm length adhesive sheet 510 is chosen; when the skin wound closing apparatus is used to close wound of which length is 25 cm˜30 cm, a 30 cm length adhesive sheet 510 is chosen.

In other embodiments, the adhesive sheet 510 side which is approaching to the other is provided with anti-bending board which is made of hard material. The skin closing apparatus 500 provided with anti-bending board is generally used to close long and straight wounds, preventing adhesive sheets 510 from distortion which has bad effect on wound closure.

The pair of threading boards 520 are symmetrically mounted the upper surface of adhesive sheets 510. Each of the threading boards 520 is provided with a plurality of threading holes 521. More particularly, in this embodiment, each of the threading boards 520 is provided with 10 threading holes 521 and threading perforations 522 corresponding to and communicated with the threading holes 521.

The threading perforations 522 facilitate the pull string thread through threading holes 521 therefore the skin wound closing apparatus can be assembled in a short time and easy to produce.

The penetrating direction of the threading holes 520 is perpendicular to the length direction of the adhesive sheets 510.

The pull string 530 is set to symmetrically thread through all the threading holes 521with the center line of the pair of adhesive sheets 510 as symmetry axis and alternately threads through the pair of adhesive sheets 510. In other embodiments, the skin wound closing apparatus may include a pull string 530, two or more pull string 530 depending on the manner of pulling. In this embodiment, the number of pull strings 530 is two. In this embodiment, the pull string 530 is evenly set between the pair of adhesive sheets 510. However, in the cases of closing certain wounds (such as wound spanning joints), the pull string 530 is set dense in the middle and sparse on the sides. The material of the pull string 530 is preferably PTFE or Parylene, the diameter of which is 0.2 mm˜1.0 mm. In this embodiment, the pull string is PTFE, the diameter of which is 0.5 mm.

FIG. 20 shows the skin wound closing apparatus in use, according to the fifth embodiment of the present invention.

As shown in FIG. 20, for the convenience of description, the threading holes 521 mounted on one of the threading boards 520 are named threading hole I1 to threading hole I10 respectively and the threading holes 521 mounted on the other threading board 520 are named threading hole J1 to threading hole J10.

In this embodiment, the number of pull strings 530 is two. One of the pull strings 530 connects the two adhesive sheets 510 by threading through threading hole I1, threading cub J1, threading hole J2, threading hole I2, threading hole I3, threading hole J3, threading hole J4, threading hole I4 threading hole I5, threading hole J5, threading hole J6, threading hole I6, threading hole I7, threading hole J7, threading hole J8, threading hole I8, threading hole I9, threading hole J10 and threading hole I10 successively. The two ends of the pull string 430 are tied to the threading board 420. The two ends of the pull string 530 are tied to the threading board 520. The other pull string 530 connects the two adhesive sheets 510 by threading through threading hole J1, threading cub I1, threading hole I2, threading hole J2, threading hole J3, threading hole I3, threading hole I4, threading hole J4, threading hole J5, threading hole I5, threading hole I6, threading hole J6, threading hole J7, threading hole I7, threading hole I8, threading hole J8, threading hole J9, threading hole I10 and threading hole J10 successively. The two ends of the pull string 530 are tied to the threading board 520.

A pair of pull components 540 is provided for the operator pulling to drive adhesive sheets 510 closer to each other to close the wound. More particularly, the pull components 540 can be pull rings provided on pull strings 530 or indicative parts of the pull string 430 such as colored parts, knotted parts and etc.

More particularly, in this embodiment, the pull string components 540 are colored parts of the pull string 530. The four pull components are positioned on the parts of the pull string 530 where threads through threading hole I2 and threading hole I3. where the pull string 530 threads through threading hole I8 and threading hole I9, where the pull string 530 threads through the threading hole J2 and J3 and where the pull string 530 threads through the J8 and J9 respectively.

The string locking squares 550 are positioned on the upper surface of adhesive sheets 510 and the corresponding position of pull components 540, forming a groove through which the unreeved string passes and by which is locked.

The structure of the string locking square 550 is exactly the same as the structure showed in the fourth embodiment and is not detailed here.

In other embodiments, the string locking square 550 can be replaced by any one of clam style locker 470, snap locker 480 or turn locker 490 described in the fourth embodiment of the skin wound closing apparatus

The structures of the clam style locker snap locker and turn locker are exactly the same as the structures showed in the fourth embodiment and are not detailed here.

The instruction of the embodiment of the skin wound closing apparatus 500 is shown as below:

Position the adhesive sheets 510 at two sides of the wound respectively, and the distance between the adhesive sheets 510 is 0.5 cm˜25 cm. After the wound is closed, the distance between the adhesive sheets 510 is 0.2 cm˜2.0 cm, the specific distance depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull components 540 in the direction of the arrow showed in FIG. 19 to drive the adhesive sheets 510 closer to each other to close the wound. After the wound is closed, the operator locks the unreeved pull string pulled out by the pull component 540 with the string locking squares 550.

In the process of the wound healing, the operator can adjust the position of the pull string 530 locked by the string locking squares 550 to adjust the tightness of the pull string 530. More particularly, if the pull string is loose, pull out more pull string 530 until the tightness is suitable; if the pull string 530 is too tight, the operator can loosen a certain length of the pull string 530 from the string locking squares 550.

After the wound is healed, it is not necessary to remove the pull string 530 and the operator can separate the adhesive sheets 510 from skin directly.

The Function and Effect of the Embodiment

According to the fifth embodiment of the skin wound closing apparatus, the skin wound closing apparatus enjoys the benefits mentioned in the first embodiment. Moreover, the fifth skin wound closing apparatus includes two pull strings and each of which is provided with two pull components positioned on the top third and bottom third therefore the operator can use four pull components at the same time. Consequentially, larger type of adhesive sheets can be driven closer to each other. Compared to the first embodiment, this embodiment of the skin wound closing apparatus is more suitable to close long wounds.

### The Sixth Embodiment

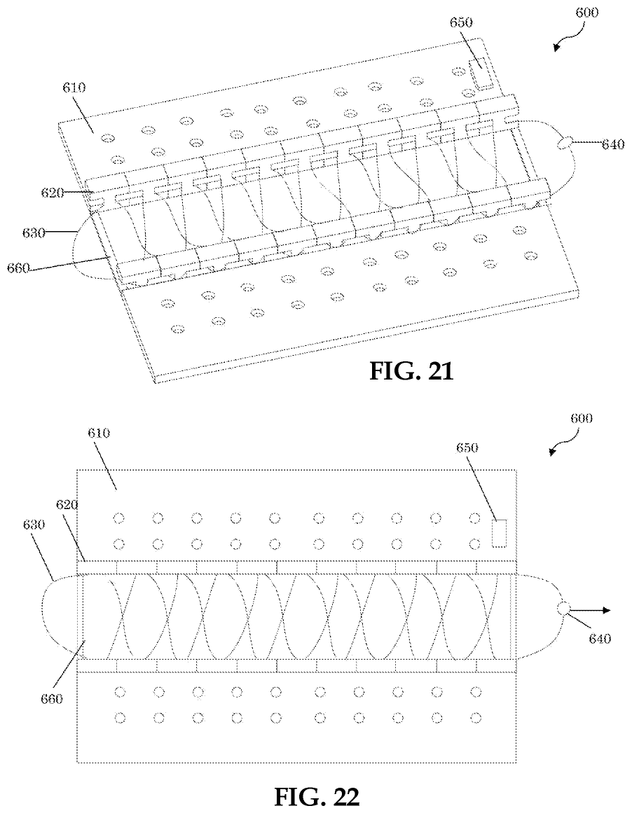

FIG. 21 and FIG. 22 respectively show schematic and top views of the skin wound closing apparatus according to the fifth embodiment of the skin wound closing apparatus.

FIG. 21-22 shows the apparatus 600 provided in this embodiment includes a pair of adhesive sheets 610, a pair of threading boards 620, a pull string 630, a pull part 640, a thread locking squares 650 and a pair of sticking strips 660.

The skin wound closing apparatus 600 provided in this embodiment can be used in closing both human wounds and animal wounds. In this embodiment, the skin wound closing apparatus 600 is used to close human wounds.

A pair of the adhesive sheets 610 is stuck to the two sides of the wound respectively and the extending length along the wound is 6 cm. The thickness of each adhesive sheet 610 is 3 mm. Each of the adhesive sheets 610 is provided with a plurality of air permeable holes facilitating patient's skin air exchange. More particularly, in this embodiment, the number of air permeable holes provided on each of the adhesive sheets 610 is 20, and are arranged in a 2×10 matrix.

In other embodiments, according to actual needs, the thickness of the adhesive sheet 610 is 1 mm˜4 mm and the extending length along the wound is 1 cm˜30 cm.

More particularly, when the skin wound closing apparatus 600 is used to close wound shorter than 1 cm, a 1 cm length adhesive sheet 510 is chosen; when the skin wound closing apparatus 600 is used to close wound of which length is 25 cm˜30 cm, a 30 cm length adhesive sheet 610 is chosen.

In other embodiments, the adhesive sheet 610 side which is approaching to the other is provided with anti-bending board which is made of hard material. The skin closing apparatus 600 provided with anti-bending board is generally used to close long and straight wounds, preventing adhesive sheets 610 from distortion which has bad effect on wound closure.

The pair of threading boards 620 are symmetrically mounted the upper surface of adhesive sheets 610. Each of the threading boards 620 is provided with a plurality of threading holes 621. More particularly, in this embodiment, each of the threading boards 620 is provided with 10 threading holes 621 and threading perforations 622 corresponding to and communicated with the threading holes 621.

The threading perforations 622 facilitate the pull string 630 threading through threading holes 621 therefore the skin wound closing apparatus 600 can be assembled in a short time and easy to produce.

The penetrating direction of the threading holes 620 is perpendicular to the length direction of the adhesive sheets 610.

The pull string 630 is set to symmetrically thread through all the threading holes 621 with the center line of the pair of adhesive sheets 610 as symmetry axis and alternately threads through the pair of adhesive sheets 610. In other embodiments, the skin wound closing apparatus 600 may include a pull string 630, two or more pull string 630 depending on the manner of pulling. In this embodiment, the number of the pull string 630 is one. In this embodiment, the pull string 630 is evenly set between the pair of adhesive sheets 610. However, in the cases of closing certain wounds (such as wound spanning joints), the pull string 630 is set dense in the middle and sparse on the sides. The material of the pull string 630 is preferably PTFE or Parylene, the diameter of which is 0.2 mm˜1.0 mm. In this embodiment, the pull string 630 is PTFE, the diameter of which is 0.5 mm.

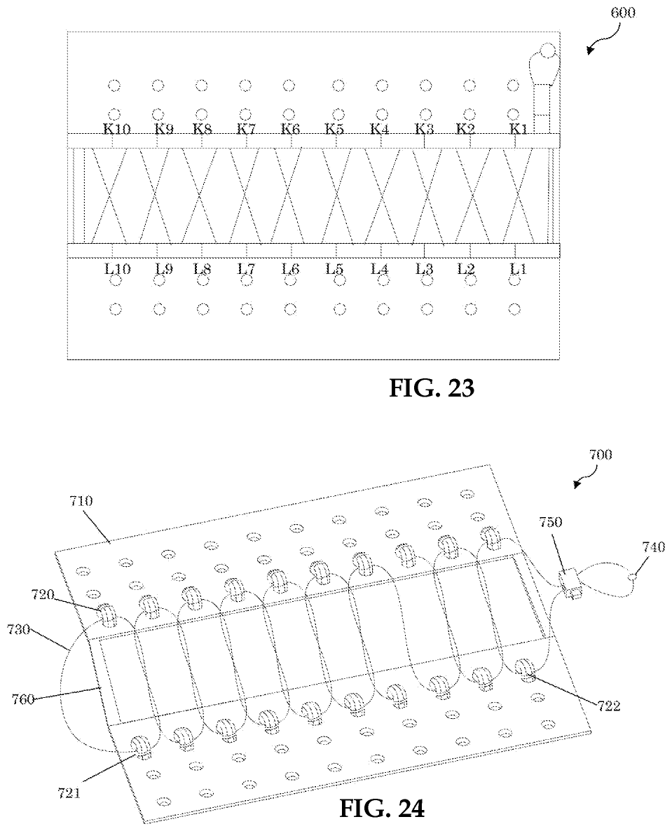

FIG. 23 shows the skin wound closing apparatus 600 in use, according to the sixth embodiment of the present invention.

As shown in FIG. 23, for the convenience of description, the threading holes 621 mounted on one of the threading boards 620 are named threading hole K1 to threading hole K10 respectively and the threading holes 621 mounted on the other threading board 620 are named threading hole L1 to threading hole L10.

The pull string 630 connects the two adhesive sheets 610 by threading through threading cube K1, threading hole L1, threading hole L2, threading hole k2, threading hole K3, threading hole L3, threading hole L4, threading hole K4, threading hole K5, threading hole L5, threading hole L6, threading hole K6, threading hole K7, threading hole L7, threading hole L8, threading hole K8, threading hole K9, threading hole L10, threading hole K10, threading hole L10, threading hole K10, threading hole K9, threading hole L9, threading hole L8, threading hole K8, threading hole K7, threading hole L7, threading hole L6, threading hole K5, threading hole L4, threading hole K4, threading hole K3, threading hole L3, threading hole L2, threading hole K2, threading hole L1 successively. The two ends of the pull string 630 are tied to the pull component 640.

A pull component 640 is provided for the operator pulling to drive adhesive sheets 610 closer to each other to close the wound. More particularly, the pull component 640 can be a pull ring provided on pull strings 530 or an indicative part of the pull string 630 such as a colored part, a knotted part and etc. More particularly, in this embodiment, the pull string component 640 is a pull ring. The two ends of the pull string 630 are tied to the pull component 640.

A string locking square 650 is positioned on the upper surface of adhesive sheets 610 and the corresponding position of pull components 640, forming a groove through which the unreeved string passes and by which is locked. The unreeved string is the part of string pulled out by the pull component 640 to drive the adhesive sheets 610 closer to each other to close the wound. The unreeved pull string can be locked on thread locking square 650 in advance and can also be locked on the thread locking square 650 after the wound is closed. In this embodiment, the operator locks the unreeved pull string 630 after the wound is closed.

The structure of the string locking square 650 is exactly the same as the structure showed in the fourth embodiment and is not detailed here.

In other embodiments, the string locking square 650 can be replaced by any one of clam style locker 470, snap locker 480 or turn locker 490 described in the fourth embodiment of the skin wound closing apparatus

The structures of the clam style locker, snap locker and turn locker are exactly the same as the structures showed in the fourth embodiment and are not detailed here.

The instruction of the embodiment of the skin wound closing apparatus 600 is shown as below:

Position the adhesive sheets 610 at two sides of the wound respectively, and the distance between the adhesive sheets 610 is 0.5 cm˜25 cm. After the wound is closed, the distance between the adhesive sheets 610 is 0.2 cm˜2.0 cm, the specific distance depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull component 640 in the direction of the arrow showed in FIG. 22 to drive the adhesive sheets 610 closer to each other to close the wound. After the wound is closed, the operator locks the unreeved pull string pulled out by the pull component 640 with the string locking square 650.

In the process of the wound healing, the operator can adjust the position of the pull string 630 locked by the string locking square 650 to adjust the tightness of the pull string 630. More particularly, if the pull string is loose, pull out more pull string 630 until the tightness is suitable; if the pull string 530 is too tight, the operator can loosen a certain length of the pull string 630 from the string locking square 650.

After the wound is healed, it is not necessary to remove the pull string 630 and the operator can separate the adhesive sheets 610 from skin directly.

The Function and Effect of the Embodiment

According to the sixth embodiment of the skin wound closing apparatus, the skin wound closing apparatus enjoys the benefits mentioned in the first embodiment. Moreover, the six skin wound closing apparatus includes one pull string and the two ends of which are connected to the pull ring therefore the operator can use the skin wound closing apparatus with one hand to drive the adhesive sheets closer to each other to close the wound. Compared to the skin wound closing apparatus provided in the first embodiment, the skin wound closing apparatus provided in this embodiment is more suitable single use, especially suitable for individual personal self-rescue in field, on the battlefield and in similar situations.

### The Seventh Embodiment

FIG. 24 and FIG. 25 respectively shows schematic and top views of the skin wound closing apparatus.

FIG. 24-25 shows the apparatus 700 provided in this embodiment includes a pair of adhesive sheets 710, a plurality pair of threading cubes 720, two pulls string 730, a pair of pull components 140, a spring loaded locker 750 and a pair of sticking strips 760.

The skin wound closing apparatus provided in this embodiment can be used in closing both human wounds and animal wounds. In this embodiment, the apparatus is used to close human wounds.

A pair of the adhesive sheets 710 is stuck to the two sides of the wound respectively and the extending length along the wound is 6 cm. The thickness of each adhesive sheet 710 is 3 mm. Each of the adhesive sheets is provided with a plurality of air permeable holes facilitating skin air exchange. More particularly, in this embodiment, the number of air permeable holes provided on each of the adhesive sheets 710 is 20, arranged in a 2×10 matrix.

In other embodiments, according to actual needs, the thickness of the adhesive sheet 710 is 1 mm˜4 mm and the extending length along the wound is 1 cm˜30 cm.

More particularly, when the skin wound closing apparatus is used to close wound shorter than 1 cm, a 1 cm length adhesive sheet 710 is chosen; when the skin wound closing apparatus is used to close wound of which length is 25 cm˜30 cm, a 30 cm length adhesive sheet is chosen.

In other embodiments, the adhesive sheet 710 side which is approaching to the other is provided with anti-bending board which is made of hard material. The skin closing apparatus 700 provided with anti-bending board is generally used to close long and straight wound, preventing adhesive sheets 710 from distortion which has bad effect on wound closure.

Each pair of threading cubes 720 are symmetrically mounted the upper surface of adhesive sheets 710. All threading cubes 720 mounted on the same adhesive sheet 710 are line up in a row along the wound. More particularly, in this embodiment, the number of threading cubes 720 mounted the same adhesive sheets 710 is 10, lined up in a row along the wound and the distance between the neighboring two threading cubes mounted on the same adhesive sheet is 5 mm.

Each of the threading cubes 720 is arc shape provided a threading hole 721 and a threading perforation 722 communicated with the threading hole 121.

The threading perforation 722 facilitates pull string 730 quickly threads through the threading hole 721, so that the skin wound closing apparatus 100 is quickly assembled and easy to produce.

The penetrating direction of the threading holes of the threading cubes 720 mounted on the same adhesive sheet 710 is parallel to the length direction of the adhesive sheet 710.

The pull string 730 is set to symmetrically thread through all the threading holes 721 with the center line of the pair of adhesive sheets 710 as symmetry axis and alternately thread through the pair of adhesive sheets 710. In other embodiments, the skin wound closing apparatus 700 may include a pull string 730, two and more pull strings 730. In this embodiment, the number of pull strings 730 is two. In this embodiment, pull strings 730 are evenly set between the pair of adhesive sheets 110. However, in the cases of closing certain wounds (such as wound spanning joints), the pull strings 730 are set dense in the middle and sparse on the sides. The material of the pull string 730 is preferably PTFE or Parylene, the diameter of which is 0.2 mm˜1.0 mm. In this embodiment, the pull strings 730 are PTFE, the diameter of which is 0.5 mm.

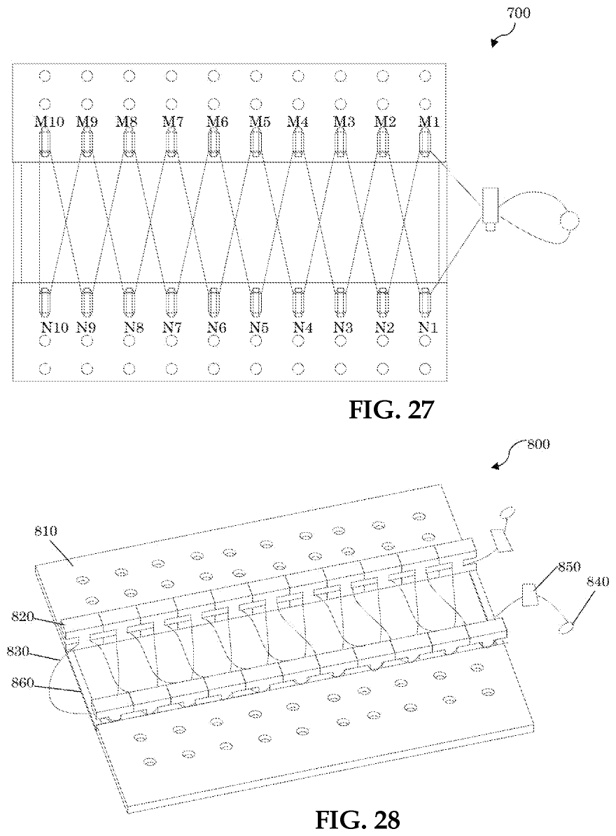

FIG. 27 shows the skin wound closing apparatus 700 in use, according to the seventh embodiment of the present invention.

As shown in FIG. 27, for the convenience of description, the threading cubes 120 mounted on one of the adhesive sheets 710 are named threading cube M1 to threading cube M10 respectively and the threading cubes mounted on the other adhesive sheet 710 are named threading cube N1 to threading cube N10.

In this embodiment, the number of the pull string 730 is one. The pull string 730 connects the two adhesive sheets 710 by threading through threading cube M1, threading cube N2, threading cube M3, threading cube N4, threading cube M5, threading cube N6. threading cube M7, threading cube N8, threading cube M9, threading cube N10 threading cube M10, threading cube N9, threading cube M8, threading cube N7, threading cube M6, threading cube N5, threading cube M4, threading cube N3, threading cube M2, threading cube N1 successively. The two ends of the pull string 630 are tied to the pull component 740, forming a closed loop.

A pair of pull components 740 is provided for operator pulling to drive adhesive sheets 710 closer to each other to close the wound. More particularly, the pull components 740 can be pull rings provided on pull strings 130 or indicative parts of pull strings such as colored parts, knotted parts and etc. More particularly, in this embodiment, pull string components 140 are pull rings. The two ends of the pull string 730 is tied on the pull component 740.

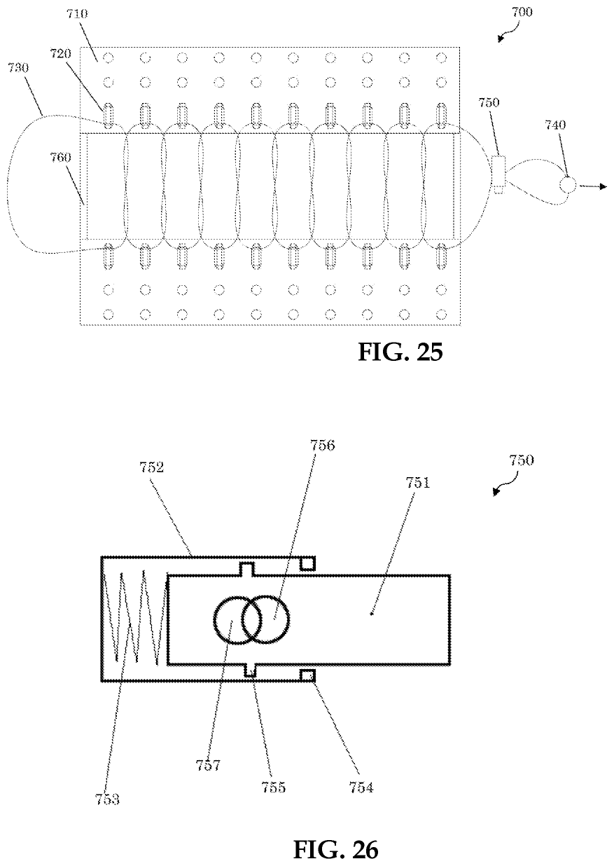

FIG. 26 is a schematic view showing the spring loaded locker of the skin wound closing apparatus, according to the seventh embodiment of the present invention.

As shown in FIG. 26, the spring loaded locker 750 includes a fixing rod 751, a shell 752, a spring 753, a first position limiter 754 and a second position limiter 755.

The fixing rod 751 is provided with a penetrating string locking perforation 756.

The shell 752 sleeves with the fixing rod 751 and is provided with a penetrating perforation 757 for the pull string 730 passing through and a fixing rod hole holding the fixing rod 751. The direction of the string locking perforation 756 is the same as the direction of the penetrating perforation 757.

The spring is positioned in the inner of the shell 752 connecting the fixing rod 751 and the shell 752. when the spring 753 is in the natural state, the string locking perforation 756 is not communicated with the penetrating perforation 757; at least when the spring 753 is in a specific compressed state, the string locking perforation 756 is communicated with the penetrating perforation 757; when the unreeved string passes both the string locking perforation 757 and the penetrating perforation 756, and the spring 753 is in the natural state, the pull string 730 is locked by the spring-loaded locker 750.

The first position limiter 754 is positioned on the hole. The second position limiter 755 is positioned on the outer surface of the fixing rod 751 in the shell 753. By the cooperation of the first position limiter 754 and the second position limiter 755, the fixing rod 751 is prevented to fall out of the shell 752. The first position limiter 751 is integrally formed with the shell 752 and the second position limiter 752 is integrally formed with the fixing rod 751.

A pair of sticking strips 760 is stuck to the two sides of the adhesive sheet 710 respectively so that the adhesive sheets 710 are mounted on skin in a parallel manner. The length and width of the sticking strips 760 are set according to actual demands. The sticking strips 760 are made of soft materials and more particularly, in this embodiment, the sticking strips 760 are soft strips.

The instruction of the embodiment of the skin wound closing apparatus 700 is shown as below:

Position the adhesive sheets 710 at two sides of the wound respectively, and the distance between the adhesive sheets 710 is 0.5 cm˜25 cm. After the wound is closed, the distance between the adhesive sheets 710 is 0.2 cm˜2.0 cm, and the distance depends on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull components 740 to drive the adhesive sheets 710 closer to each other to close the wound. At the moment, the distance between two adhesive sheets 710 is 0.2 cm˜2.0 cm. After the wound is closed, the operator locks the part of pull string of the pull component 740 with the spring loaded locker 750.

In the process of the wound healing, the operator can adjust the position of the pull string 730 locked by the spring loaded locker 150 to adjust the tightness of the pull string 730. More particularly, if the pull string 730 is loose, press the fixing rod 751 in the direction toward the shell 752 and pull the pull component 740 until the tightness is suitable; if the pull string 730 is too tight, the operator can press the fixing rod 751 in the direction toward the shell 75 and move the spring loaded locker 750 toward the direction of the adhesive sheets 710 until the tightness is suitable.

After the wound is healed, it is not necessary to remove the pull string 730 and the operator can separate the adhesive sheets 710 from skin directly.

The Function and Effect of the Embodiment

According to the seventh embodiment of the skin wound closing apparatus, the skin wound closing apparatus includes a spring loaded locker therefore the unreeved string pulled out by the pull component is locked by the spring loaded locker. Consequentially, a stable wound closure is realized.

Furthermore, according to the seventh embodiment of the skin wound closing apparatus, the skin wound closing apparatus adopts one pull string. The two ends of the pull string was tied to the pull ring served as pull component. Consequentially, the operator can adjust the tightness in a very convenient manner.

### The Eighth Embodiment

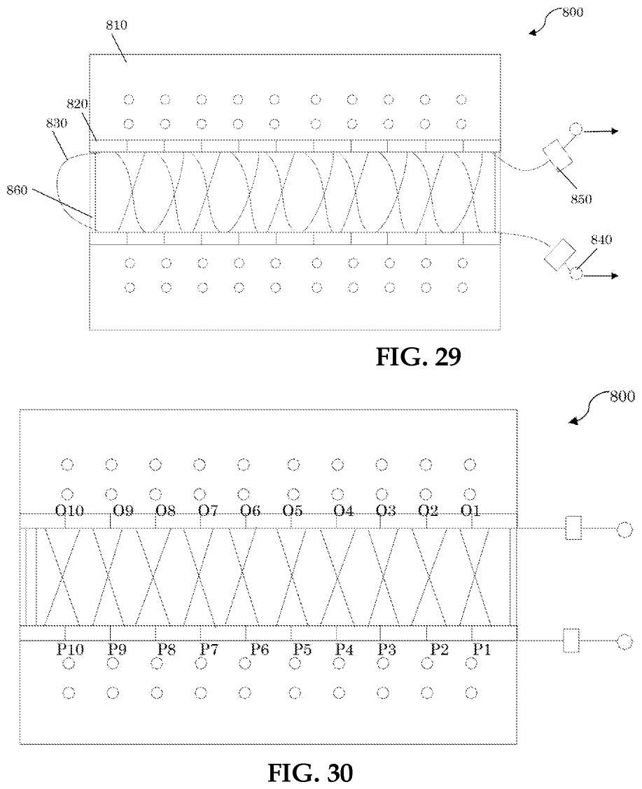

FIG. 28 and FIG. 29 respectively show schematic and top views of the skin wound closing apparatus according to the fifth embodiment of the skin wound closing apparatus.

FIG. 28-29 show the apparatus 800 provided in this embodiment includes a pair of adhesive sheets 810, a pair of threading boards 820, a pull string 830, a pull part 840, a pair of adhesive strips 850 and a pair of sticking strips 860.

The skin wound closing apparatus 800 provided in this embodiment can be used in closing both human wounds and animal wounds. In this embodiment, the skin wound closing apparatus 800 is used to close human wounds.

A pair of the adhesive sheets 810 is stuck to the two sides of the wound respectively and the extending length along the wound is 6 cm. The thickness of each adhesive sheets 810 is 3 mm. Each of the adhesive sheets 810 is provided with a plurality of air permeable holes facilitating skin air exchange. More particularly, in this embodiment, the number of air permeable holes provided on each of the adhesive sheets 810 is 20, and are arranged in a 2×10 matrix.

In other embodiments, according to actual needs, the thickness of the adhesive sheet 810 is 1 mm˜4 mm and the extending length along the wound is 1 cm˜30 cm.

More particularly, when the skin wound closing apparatus 800 is used to close wound shorter than 1 cm, a 1 cm length adhesive sheet 810 is chosen; when the skin wound closing apparatus 800 is used to close wound of which length is 25 cm˜30 cm, a 30 cm length adhesive sheet 810 is chosen.

In other embodiments, the adhesive sheet 810 side which is approaching to the other is provided with anti-bending board which is made of hard material. The skin closing apparatus 800 provided with anti-bending board is generally used to close long and straight wounds, preventing adhesive sheets 810 from distortion which has bad effect on wound closure.

The pair of threading boards 820 are symmetrically mounted the upper surface of adhesive sheets 810. Each of the threading boards 820 is provided with a plurality of threading holes 821. More particularly, in this embodiment, each of the threading boards 820 is provided with 10 threading holes 821 and threading perforations 822 corresponding to and communicated with the threading holes 821.

The threading perforations 822 facilitate the pull string 810 threading through threading holes 621 therefore the skin wound closing apparatus 800 can be assembled in a short time and easy to produce.

The penetrating direction of the threading holes 820 is perpendicular to the length direction of the adhesive sheets 810.

The pull string 830 is set to symmetrically thread through all the threading holes 821 with the center line of the pair of adhesive sheets 810 as symmetry axis and alternately threads through the pair of adhesive sheets 810. In other embodiments, the skin wound closing apparatus 800 may include a pull string 830, two or more pull string 830 depending on the manner of pulling. In this embodiment, the number of the pull string 830 is one. In this embodiment, the pull string 830 is evenly set between the pair of adhesive sheets 810. However, in the cases of closing certain wounds (such as wound spanning joints), the pull string 830 is set dense in the middle and sparse on the sides. The material of the pull string 830 is preferably PTFE or Parylene, the diameter of which is 0.2 mm˜1.0 mm. In this embodiment, the pull string 830 is PTFE, the diameter of which is 0.5 mm.

FIG. 30 shows the skin wound closing apparatus 800 in use, according to the sixth embodiment of the present invention.

As shown in FIG. 23, for the convenience of description, the threading holes 821 mounted on one of the threading boards 820 are named threading hole O1 to threading hole O10 respectively and the threading holes 821 mounted on the other threading board 820 are named threading hole P1 to threading hole P10.

In this embodiment, the number of the pull string 830 is one. The pull string 830 connects the two adhesive sheets 810 by threading through threading hole O1, threading hole P1, threading hole P2, threading hole O2, threading hole O3, threading hole P3, threading hole P4, threading hole O4, threading hole O5, threading hole P5, threading hole P6, threading hole O6, threading hole O7, threading hole P7, threading hole P8, threading hole O9, threading hole P10, threading hole O10, threading hole O9, threading hole P9, threading hole P8, threading hole O8, threading hole O7, threading hole P7, threading hole P6, threading hole O6, threading hole O5, threading hole P5, threading hole P4, threading hole O4, threading hole O3, threading hole P3, threading hole P2, threading hole O2, threading hole P1 successively. The two ends of the pull string is tied on a pair of the pull component 840.

The pull components 640 are provided for the operator pulling to drive adhesive sheets 810 closer to each other to close the wound. More particularly, the pull components 840 can be pull rings provided on pull strings 530 or indicative parts of the pull string 830 such as colored parts, knotted parts and etc.

More particularly, in this embodiment, the pull string components 840 are pull rings. The two ends of the pull string 830 are tied to the pull components 840.

The unreeved string is locked by the adhesive strip 850 on the skin or the adhesive sheets 810. The adhesive side of the adhesive strip 850 is provided with release paper which prevent the adhesive strip 850 from sticking to other items when is not used. When the adhesive strip 850 is stuck to the adhesive sheets 810, the adhesive strip can be any kind of adhesive strip; when the adhesive strip is stuck to skin, the adhesive strip 850 must be medical adhesive strip. In this embodiment, the operator locks the unreeved pull string 830 after the wound is closed. In other embodiments, the unreeved pull string can be locked on skin by the adhesive strip 850 in advance and can also be locked on skin by the adhesive strip 850 after the wound is closed.

A pair of sticking strips 860 is stuck to the two sides of the adhesive sheet 810 respectively so that the adhesive sheets 810 are mounted on skin in a parallel manner. The length and width of the sticking strips 860 are set according to actual demands. The sticking strips 860 are made of soft materials and more particularly, in this embodiment, the sticking strips 860 are soft strips.

The instruction of the embodiment of the skin wound closing apparatus 600 is shown as below:

Position the adhesive sheets 810 at two sides of the wound respectively, and the distance between the adhesive sheets 810 is 0.5 cm˜25 cm. After the wound is closed, the distance between the adhesive sheets 810 is 0.2 cm˜2.0 cm, the specific distance depending on the length of the wound. When the length of wound is 1 cm˜3 cm, the distance before the wound is closed is 8 mm and after the wound is closed, the distance is 3 mm; when the length of wound is 3 cm˜5 cm, the distance before the wound is closed is 10 mm, and after the wound is closed, the distance is 5 mm; when the length of the wound is 5 cm˜10 cm, the distance before the wound is closed is 12 mm and after the wound is closed, the distance is 8 mm; when the length of the wound is over 20 cm, the distance before the wound is closed is 20 mm and after the wound is closed is 12 mm. Pull the pull component 840 in the direction of the arrow showed in FIG. 29 to drive the adhesive sheets 810 closer to each other to close the wound. At the moment, the distance between the two adhesive sheets is 1.0 cm˜2.0 cm. After the wound is closed, the operator removes the release paper and locks the unreeved pull string pulled out by the pull component 840 on skin with the adhesive strips 850.

In the process of the wound healing, the operator can adjust the position of the pull string 830 locked by the string locking square 850 to adjust the tightness of the pull string 830. More particularly, remove the adhesive strips 850 and adjust the pull component 840 to a suitable tightness and the locks the unreeved string with the adhesive strips 850.

After the wound is healed, it is not necessary to remove the pull string 830 and the operator can separate the adhesive sheets 810 from skin directly.

The Function and Effect of the Embodiment

According to the eighth embodiment of the skin wound closing apparatus, the skin wound closing apparatus enjoys the benefits mentioned in the first embodiment. Moreover, the skin wound closing apparatus provided with adhesive strips therefore the skin wound closing apparatus locks the unreeved string pulled out by the pull component in a quick and convenient manner. Consequentially, the skin wound closing apparatus close the wound stably, is convenient to operate and is suitable to promote in medical institutions.

The use of any and all examples or exemplary language (e.g., “such as”) provided herein, is intended merely to better illuminate embodiments of the invention and does not pose a limitation on the scope of the invention unless otherwise claimed.

What is claimed is:

1. A skin wound closing apparatus for the operator to close wounds, comprising:
a pair of adhesive sheets, which are capable to stick on both sides of said wound respectively;
at least a pair of threading components, each pair of said threading components are symmetrically positioned on the upper surface of said adhesive sheets respectively, and each of said threading components is provided with threading holes;
at least a pull string, which symmetrically threads through all said threading holes in a manner that is symmetric to the center line of said pair of adhesive sheets and alternately threads through said pair of adhesive sheets;
at least a pull component, by which the operator pulls to drive said pair of adhesive sheets closer to each other so that said wound is closed; and
a pull string locking component, by which the unreeved string is locked, said unreeved string is the part of string pulled out by said pull string component when said pair of adhesive sheets are driven closer to each other to close said wound.

2. The skin wound closing apparatus of claim 1, wherein said threading components are threading boards, which are provided with a plurality of threading holes.

3. The skin wound closing apparatus of claim 2, wherein each of said threading boards is provided with a threading perforation corresponding to and communicated with said threading hole,
said threading perforation enables said pull string threading through said threading hole,
the penetrating direction of said threading hole is perpendicular to the length direction of said adhesive sheets.

4. The skin wound closing apparatus of claim 1, wherein said threading components are threading cubes, each of which is provided with a threading hole.

5. The skin wound closing apparatus of claim 4, wherein each of said adhesive sheets is arranged with a plurality of threading cubes which are lined up in a row along said wound,
each of said threading cubes is provided with said threading perforation communicated with said threading hole, which facilitates said pull string threading through said threading holes,
the penetrating direction of said threading perforation is parallel to the length direction of said adhesive sheets.

6. The skin wound closing apparatus of claim 1, wherein said pull string locking components are string locking squares, which are mounted on said adhesive sheets forming a groove through which said unreeved pull string passes and by which is locked on said string locking squares.

7. The skin wound closing apparatus of claim 1, wherein said pull string locking component is a clampstyle locker,
said clampstyle locker is provided with a groove for said pull string passing through,
said unreeved pull string passes through said groove and is locked by said clampstyle locker.

8. The skin wound closing apparatus of claim 1, wherein said pull string locking component is a snap locker comprising:
a snap locker base, which is mounted on said adhesive sheets, provided with a groove; and
a button, provided with a tongue matching with said groove,
said unreeved pull string passes through said groove and is compressed and locked in said groove by engaging said button with said snap locker base.

9. The skin wound closing apparatus of claim 1, wherein said pull string locking component is a turn locker comprising:
a turn locker base, mounted on said adhesive sheets and provided with a groove and furthermore said groove provided with screw thread;
a rotating component provided with a screw threaded rod of which the screw thread matches with the screw thread on said groove.
said unreeved string passes through said groove, by engaging said rotating component with said turn locker base said unreeved string is locked in said groove.

10. The skin wound closing apparatus of claim 1, wherein said pull string component is a spring locker comprising:
a shell mounted on said adhesive sheets;
a spring arranged in said shell;
a convex square, one end of which is connected with said spring and the other end extends out of said shell;
a locking square, which is positioned on the same adhesive sheet as said shell positioned and by engaging said locking square with said convex square, the pull string is locked.

11. The skin wound closing apparatus of claim 1, wherein said pull string locking component is an elastic needle locker comprising:
an elastic needle fixing square, which is mounted on said adhesive sheets;
a stopper, provided with sliding groove; and
an elastic needle, one end of which is positioned on said elastic needle fixing square, the other end of which engages with said stopper to lock said unreeved string in said sliding groove,
said elastic needle and said pull string are at an obtuse angle when pulled by the operator.

12. The skin wound closing apparatus of claim 1, said pull string component is a spring-loaded locker comprising:
a fixing rod provided with a penetrating string locking perforation;
a shell sleeving with said fixing rod and provided with a penetrating perforation for said pull string passing through and a fixing rod hole holding said fixing rod; and
a spring positioned in the inner of said shell connecting said fixing rod and said shell,
the direction of said string locking perforation is the same as the direction of the penetrating perforation,
when said spring is in the natural state, said string locking perforation is not communicated with said penetrating perforation,
at least when said spring is in a specific compressed state, said string locking perforation is communicated with said penetrating perforation,
when said unreeved string passes through both said string locking perforation and said penetrating perforation, and said spring is in the natural state, said pull thread is locked by said spring-loaded locker.

13. The skin wound closing apparatus of claim 12, wherein said spring-loaded locker comprising:
a first position limiter positioned on said fixing rod hole; and
a second position limiter positioned on the outer side of said fixing rod in said shell, by the cooperation of said first position limiter and said second position limiter said fixing rod is prevented from falling out from said shell.

14. The skin wound closing apparatus of claim 1, wherein said pull string components are adhesive strips,
said adhesive strips lock said unreeved string on said adhesive sheets or skin.

15. The skin wound closing apparatus of claim 1, wherein the number of said pull string is one,
a pair of pull components are positioned on the two ends of said pull string respectively,
the operator pulls said pull components in the direction parallel to the length direction of said adhesive sheets to drive said two adhesive sheets closer to each other, so that said wound is closed.

16. The skin wound closing apparatus of claim 1, wherein the number of the pull string is two,
two ends of said pull strings are positioned on the ends of said adhesive sheets respectively,
said pull components are positioned at the middle of each of said adhesive sheets and are positioned on said pulling strings,
the operator pulls said pulling part at the opposite direction perpendicular to the extension of said wound to drive said two adhesive sheets closer to each other, so that said wound is closed.

17. The skin wound closing apparatus of claim 1, wherein two sticklers are mounted on the two sides of said adhesive sheet, so that said adhesive sheets are stuck on the skin in a parallel manner.

18. The skin wound closing apparatus of claim 1, wherein said pull components is a pull ring or an indicative part of said pull string.

19. The skin wound closing apparatus of claim 1, wherein after said skin wound closing apparatus are stuck to the skin, and before the wound closing operation, the distance between said two adhesive sheets is 0.5 cm˜3.0 cm;
after the wound closing operation, the distance between said two adhesive sheets is 0.2 cm˜2.0 cm.

20. The skin wound closing apparatus of claim 1, wherein said adhesive sheets are arranged with a plurality of air permeable holes.
